(19) United States
(12) Patent Application Publication
(10) Pub. No.: US 2006/0094109 A1
(43) Pub. Date: May 4, 2006

(54) Device and method for analytical cell imaging

(75) Inventor: Michael Norman Trainer, Coopersburg, PA (US)

Correspondence Address: IMMUNICON CORPORATION, 3401 MASONS MILL ROAD, SUITE 100, HUNTINGDON VALLEY, PA, 19006, US

(73) Assignee: Immunivest Corporation

(21) Appl. No.: 10/979,388

(22) Filed: Nov. 2, 2004

(51) Int. Cl. C12M 1/34; C12Q 1/04

(52) U.S. Cl. 435/288.7; 382/133; 435/34; 435/808

(57) ABSTRACT

In patients with carcinomas tumor cells are shed into the blood, enumeration and characterization of these cells offers the opportunity to obtain a “real time” biopsy of the tumor and may improve the management of the disease. The frequency of circulating tumor cells is rare (<1 cell/ml) and technology is needed that has sufficient sensitivity and specificity to enumerate and characterize these cells. The present system was developed to provide an immunophenotype, fluorescence wave forms as well as images of immunomagnetically enriched cells. Blood volumes ranging from 7.5-30 ml are immunomagnetically enriched for epithelial cells. The sample volume is reduced to 320 μl and inserted into an analysis chamber. Upon introduction of the chamber in a magnetic field, the immunomagnetically tagged cells rise out of the sample and align between nickel lines (period 30 μm, space 15 μm) that are present on the viewing surface of the chamber. A multi laser system is used to detect the fluorescence emitted by DAPI, Phycoerythrin and Allophycocyan labeled and magnetically aligned cells. Compact disk optics are used to maintain alignment and focus of the laser beams onto nickel lines while moving the chamber. The chamber is scanned with a speed of 10 mm/sec and the entire chamber is analyzed in approximately 5 minutes. The fluorescent signals obtained from the events provide an immunophenotype similar to that of a flow cytometer. The fluorescence waveforms improve the characterization of the events and add to the classification as background, cellular debris and cells. Since the cell locations are preserved, objects that immunophenotypically classify as epithelial cells can be revisited for further analysis. Bright field and fluorescent images of the selected objects are captured to confirm that the identified objects are tumor cells.

### PRIORITY INFORMATION

This application is U.S. National Stage of PCT/US03/13842, which claims the benefit of U.S. Provisional Application No. 60/377,868 filed 3 May 2002. That application is incorporated by reference herein.

### BACKGROUND OF THE INVENTION

Automated image analysis systems have been used to reduce subjective errors in cell classification between different operators in manual methods, but such prior art systems without preliminary cell enrichment steps still inherently lack sensitivity. Several automated cell-imaging systems have been described or are commercially available for cell analysis. The system developed by Chromavision, ACIS™ or Automated Cellular Imaging System (Douglass et al., U.S. Pat. No. 6,151,405) uses calorimetric pattern recognition by microscopic examination of prepared cells by size, shape, hue and staining intensity as observed by an automated computer controlled microscope and/or by visual examination by a health care professional. The system uses examination of cells on microscope slides and was designed for tissue sections. The SlideScan™ or MDS™ systems of Applied Imaging Corp. (Saunders et al., U.S. Pat. No. 5,432,054) is described as an automated, intelligent microscope and imaging system that detects cells or “objects” by color, intensity, size, pattern and shape followed by visual identification and classification. In contrast to the ACIS system this system has the ability to detect fluorescent labels, which provides more capability. However, these and other currently available methodologies are not sufficiently sensitive for accurate classification and typing of rare events such as circulating tumor cells in blood.

Epithelial cells are not present in blood under normal circumstances. In patients with epithelial derived cancer (carcinomas) cancer cells can be shed into the blood. These cells are rare in peripheral blood and exhibit a large dynamic range from patient to patient. Tumor cells can be present in blood of carcinoma patients at extremely low frequencies (<10 cells/mL). Flow cytometry and/or fluorescence microscopy are analytical methods frequently used for analyzing the prepared samples. Flow cytometry has the advantage that it is sensitive and reproducible but it lacks the ability to simultaneously assess the immunophenotype and morphological features of the detected cells. Although fluorescence activated cell sorting (FACS) can be used to sort immunophenotypically identified cells it is quite a challenge to sort the rare events and preserve them for cytological evaluation. In addition, the skill level needed for the latter is prohibitive for a clinical assay. Fluorescence microscopy has the disadvantage that considerable and variable cell losses are associated with the preparation of the sample slides for microscopic analysis. However it has the advantage that a cell can be visually confirmed as having features consistent with malignancy.

An analytical system must be capable of accurately identifying as few as 1 cell while still being capable of enumerating as many as 10⁴ cells. The detection of these circulating tumor cells (CTC) is further complicated by their heterogeneity, not only in size and shape, but also in their antigen expression profile such as cytoskeletal proteins that can be present at extremely low or high copy numbers.

Accordingly, the present invention seeks to improve upon the aforementioned methodologies, and to provide simple and efficient means and methods for automated imaging of objects that can be used, for example, in conjunction with high sensitivity immunophenotyping, to permit detection, enumeration and accurate classification of rare target species, such as CTC in blood or other fluids.

### BRIEF DESCRIPTION OF THE INVENTION

The present invention is a cell analyzer that differentiates immunomagnetically cells that are aligned on Nickel lines while passing through a focused laser beam. In one preferred embodiment, a conventional CD player objective was used to focus a laser-diode onto the magnetically aligned cells. An optical focus and tracking system analogous to that used in a CD player was used to scan along the lines. The emitted fluorescence signals were projected onto, and measured by, photomultiplier tubes. The absolute and relative cell populations identified by the instrument system correlated well with the numbers obtained with a standard flow cytometer or hematology analyzer.

In further embodiments, the features of the instrument system were expanded to demonstrate the potential for rare cell analysis by building on its sensitivity to measure immunofluorescence signals. This was accomplished through the addition of the ability to revisit the events of interest, and providing bright field and fluorescent images of these objects.

A further embodiment of the invention includes a scanning mirror to deflect the illumination sources. As the objects of interest pass through illumination, a “rastered” pattern is formed across the objects, which is subsequently detected and transformed into a more detailed image of the object, creating a raster image.

### BRIEF DESCRIPTION OF THE FIGURES

FIG. 1 diagrams the optical layout of the instrument system. DCLP=dichroic long pass filter; DCSP=dichroic short pass filter; PMT=Photomultiplier Tube

FIG. 2 shows the sample chamber and magnetic design. Panel A is the sample chamber and plug to seal the chamber after filling. Panel B is the magnetic fixture that holds the sample chamber in position and move magnetically labeled objects to the upper surface of the chamber. A 3 mm space separates the two angled magnets depicted black. The magnets are yoked by steel depicted gray. Panel C is a computer simulation of magnetically labeled cells present in the chamber. The dotted lines in the chamber represent the trajectory of randomly positioned magnetically labeled cells that move to the surface under influence of the magnetic gradient. Panel D is a magnification of the simulation shown in Panel C illustrating the trajectory of magnetically labeled cells once they approach the surface of the chamber.

FIG. 3 is a photomicrograph of PC3 cells alignment between Nickel lines. The relative size and distribution of the three laser spots are indicated in the figure (magnification 200×)

FIG. 4 diagrams the configuration of laser scanning that results in rastering.

FIG. 5 shows the distribution of the cell size of circulating tumor cells. Tumor cell sizes of 1030 cells identified by a fluorescent microscope system in the blood of 8 carcinoma patients

FIG. 6 shows the results of the analysis of 7.5 ml blood sample spiked with tumor cells. Panel A shows the 2-dimensional dot plot of CK-PE versus CD45-APC: 295 tumor cells, 126 leukocytes and 11 CD45+, CK+ cells were detected in the sample. Approximately 300 cells of the prostate cell line PC3 were spiked into 7.5 ml of blood. Panel B shows an acquired waveform of the CK-PE signal. Panel C shows a bright field image of an object. Panel D shows the nuclear image of the object in panel C

FIG. 7 diagrams an alternative optical layout of the cell tracks system. DCLP=dichroic long pass filter; DCSP=dichroic short pass filter; PMT=Photomultiplier Tube

FIG. 8 shows a scanned image and the corresponding intensity profile for a static laser (A & B) and a scanning laser (C & D).

FIG. 9 diagrams the imaging subsystem (A), and shows a bright field image (B), and a laser scanned DAPI image (C & D).

FIG. 10 shows the analysis of the system (A) that has found a tumor cell candidate. The system then revisits this object and performs bright field imaging (B), scanning laser imaging (C), and combines the images into one (D).

FIG. 11 shows bright field images of objects of interest that are revisited using the encoder position of the Y-stage recorded with each data point.

### DETAILED DESCRIPTION OF THE INVENTION

Herein, various terms that are well understood by those of ordinary skill in the art are used. The intended meaning of these terms does not depart from the accepted meaning.

This invention provides devices and methods that permit the application of novel imaging capabilities to such systems as the CellTracks™ cell analysis system as described by Tibbe et al. (Nature Biotech. 17, 1210-13, 1999). Briefly, in a preferred embodiment of the invention, after magnetic collection and enrichment from blood, the magnetically labeled cells are aligned along ferromagnetic lines of nickel (Ni) and are scanned by a laser focused by means of a conventional objective lens such as from a compact disk player. Since the cells have been selectively stained with one or more fluorescent labels, the measured fluorescence emissions and the intensities can be used to identify or classify the cell type.

Epithelium derived tumor cells in peripheral blood are extremely rare but can be present in the blood of cancer patients. During analysis, the certainty that an event present in a biological sample is an epithelial cell with the assumed characteristics diminishes with the number of events in the analysis gate. Additional and preferably independent information on the individual events aids in the correct classification of the event as an epithelium derived tumor cell. As described hereinbelow, epithelial cells will be immunomagnetically selected from 7.5 mL of blood and magnetically aligned in a sample chamber between a series of parallel thin film nickel lines. The CD head scans along all nickel lines and captures the fluorescence signals of the objects between the lines. Objects that immunophenotypically classify as epithelial tumor cells are revisited for imaging to determine if the identified objects indeed classify as epithelial tumors cells or as debris derived from epithelial cells.

The term “target bioentities” as used herein refers to a wide variety of materials of biological or medical interest and can be distinguished from “non-target” materials that are present in the specimen. Examples include hormones, proteins, peptides, lectins, oligonucleotides, drugs, chemical substances, nucleic acid molecules, (e.g., RNA and/or DNA) and particulate analytes of biological origin, which include bioparticles such as cells, viruses, bacteria and the like. In a preferred embodiment of the invention, rare cells, such as fetal cells in maternal circulation, or circulating cancer cells may be efficiently isolated from non-target cells and/or other bioentities, using the apparatus and methods of the present invention.

The terms “biological specimen” or “biological sample” may be used interchangeably, and refer to a small potion of fluid or tissue taken from a human test subject that is suspected to contain biological entities (or bioentities) of interest, and is to be analyzed. A biological specimen refers to the fluidic portion, the cellular portion, and the portion containing soluble material. Biological specimens or biological samples include, without limit bodily fluids, such as peripheral blood, tissue homogenates, nipple aspirates, colonic lavage, sputum, bronchial (alveolar) lavage, pleural fluids, peritoneal fluids, pericardial fluids, urine, and any other source of cells that is obtainable from a human test subject. An exemplary tissue homogenate may be obtained from the sentinel node in a breast cancer patient. Biological entities refer to objects of interest, as would be understood from the previous description.

The term “determinant”, when used in reference to any of the foregoing target bioentities, refers broadly to chemical mosaics present on macromolecular antigens that often induce an immune response. Determinants may also be used interchangeably with “epitopes”. A “biospecific ligand” or a “biospecific reagent,” used interchangeably herein, may specifically bind determinants. A determinant refers to that portion of the target bioentity involved in, and responsible for, selective binding to a specific binding substance (such as a ligand or reagent), the presence of which is required for selective binding to occur. In fundamental terms, determinants are molecular contact regions on target bioentities that are recognized by agents, ligands and/or reagents having binding affinity therefore, in specific binding pair reactions.

The term “specific binding pair” as used herein includes antigen-antibody, receptor-hormone, receptor-ligand, agonist-antagonist, lectin-carbohydrate, nucleic acid (RNA or DNA) hybridizing sequences, Fc receptor or mouse IgG-protein A, avidin-biotin, streptavidin-biotin and virus-receptor interactions.

The term “detectably label” is used to herein to refer to any substance whose detection or measurement, either directly or indirectly, by physical or chemical means, is indicative of the presence of the target bioentity in the test sample. Representative examples of useful detectable labels, include, but are not limited to the following: molecules or ions directly or indirectly detectable based on light absorbance, fluorescence, reflectance, light scatter, phosphorescence, or luminescence properties; molecules or ions detectable by their radioactive properties; molecules or ions detectable by their nuclear magnetic resonance or paramagnetic properties. Included among the group of molecules indirectly detectable based on light absorbance or fluorescence, for example, are various enzymes which cause appropriate substrates to convert (e.g., from non-light absorbing to light absorbing molecules, or from non-fluorescent to fluorescent molecules). Analysis can be performed using any of a number of commonly used platforms, including multiparameter flow cytometry, immunofluorescent microscopy, laser scanning cytometry, bright field base image analysis, capillary volumetry, spectral imaging analysis, manual cell analysis, CellSpotter® analysis, CellTracks™ analysis, and automated cell analysis.

The phrase “to the substantial exclusion of” refers to the specificity of the binding reaction between the biospecific ligand or biospecific reagent and its corresponding target determinant. Biospecific ligands and reagents have specific binding activity for their target determinant yet may also exhibit a low level of non-specific binding to other sample components.

The present system was designed to identify rare cells. The term “rare cells” is defined herein as cells that are not normally present in biological specimens, but may be present as an indicator of an abnormal condition, such as infectious disease, chronic disease, injury, or pregnancy. Rare cells also refer to cells that may be normally present in biological specimens, but are present with a frequency several orders of magnitude less than cells typically present in a normal biological specimen. The detection and enumeration procedure used herein is further described in U.S. Pat. No. 6,365,362, which is incorporated by reference.

The optics of the present system includes an illumination source and a detector. As an example, the illumination means may be narrow-spectrum lasers or LEDs. It may also be a broad-spectrum white light source, where light is passed through narrow spectrum filters to achieve the desired wavelengths. The detection components consist of two-dimensional arrays of detector elements. Examples of such a detector array include PMTs and CCDs. However, persons skilled in the art will appreciate that other illumination and detection means may be used in this invention.

A sample preparation process involving immunomagnetic selection, separation, and staining of cells from blood volumes ranging from 1-30 mL precedes the analysis of the sample by the present system. Cells were transferred to a sample chamber (described in U.S. Pat. No. 6,136,182 and US 2002/0109838, incorporated by reference herein) and magnetically aligned between 90 parallel 15 □m-wide Nickel lines on the inside surface of the sample chamber, as described in U.S. Pat. No. 5,985,153, which is incorporated by reference herein. The CD head scans along all Nickel lines with an illuminating means in less than 5 minutes, capturing the fluorescence signals of the objects between the lines with a detection means. Cells maintain their position by magnetic force, which creates the possibility to revisit cells of interest. The imaging techniques used in this invention are based on those described in the commonly owned application PCT/US02/00203, which is incorporated by reference herein.

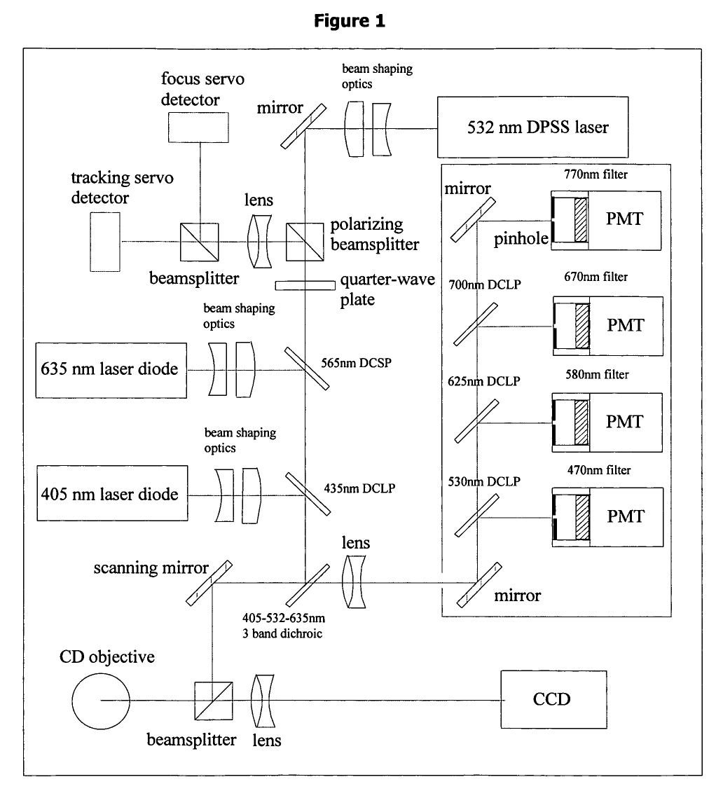

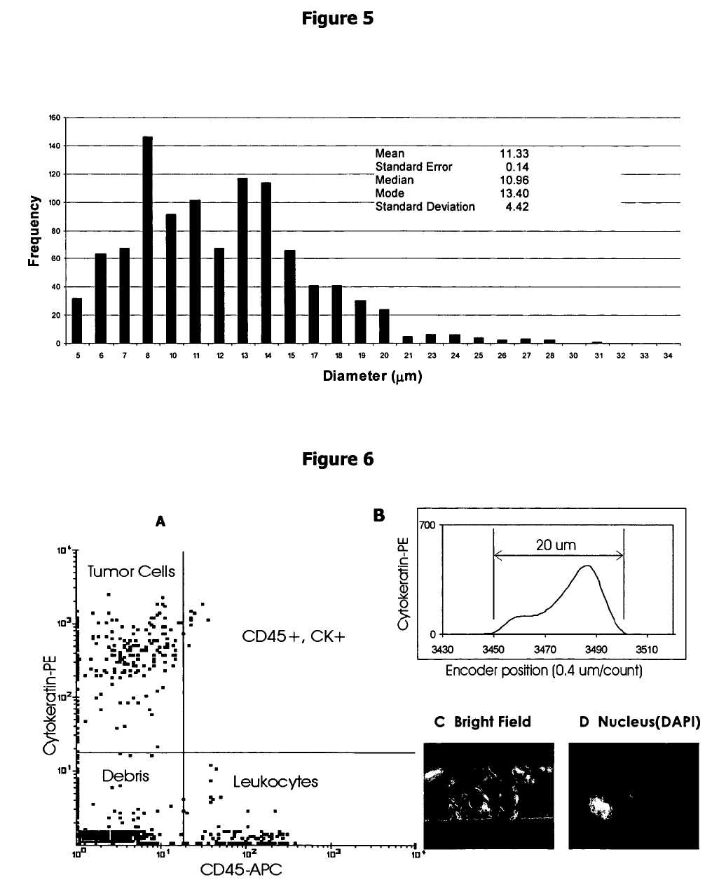

Low noise, high signals, and minimal fluorescence emission spectral overlap of the fluorescence probes are essential for rare cell detection. The system was equipped with 405 nm, 532 nm and 635 nm lasers for excitation of DAPI, PE and APC respectively. To confirm or reject an identified object as a cell, the object of interest is revisited and a violet laser diode is used to obtain an image of the DAPI stained nucleus (FIG. 5D). To overcome the laser speckle, a scanning mirror was used to obtain a homogenous illumination. The amount of DNA in the object can be quantified by measuring the DAPI emission with the photomultiplier, FIG. 1. The high extinction coefficient and quantum yield and the large Stokes shifts of PE makes it an excellent choice as the fluorochrome for labeling the cytokeratin antibody that identifies the cytoskeletal proteins in the epithelial cells. A 532 nm light source was chosen to excite PE as it provides a 6-fold increase in signal to noise ratio as compared to 488 nm excitation. In the present invention, the time a cell is exposed to the laser spot is orders of magnitude larger compared to a flow cytometer and more photons can thus be collected which explains the higher sensitivity of the system. Although far from optimal, PE can be excited by the 405 nm laser line. However, we have not yet compared the signal to noise ratio with both excitation sources at different scanning speeds. The 635 nm light source was chosen to excite APC thereby avoiding any overlap between the excitation and emission spectra of PE. The CD45-APC permits the discrimination of the leukocytes carried over through the enrichment procedure from the epithelial cells. More importantly it avoids identifying the leukocytes that non-specifically bind to CK-PE as epithelial cells. Leukocytes can be attached to epithelial derived tumor cells in blood. This phenomenon would result in a relevant CK+ and CD45+ object that can be distinguished from the non-relevant objects by the waveform of the fluorescent signals in concert with the images of the cells.

In summary, the immunophenotype, spatial distributions of the digitized fluorescence signals and the bright field and fluorescence images, measured in the system of the present invention, enables the identification of epithelial cells present at low frequency in blood and further enables sub classification of these cells such as intact, damaged or epithelial cell debris. While in this study carcinoma cells lines were used to demonstrate performance of the instrument, it is clear that the instrument can be used for a variety of cell analyses including the detection of other rare events, such as endothelial cells in blood, fetal cells in maternal blood, bacteria in blood or other fluids. The sample preparation process can be modified to be specific to any target of interest (by ferrofluid selection) and the staining of cells would have to be compatible with the laser wavelength and filter selections established within the system.

The system of the present invention was calibrated using 6 □m Deep Red fluorescent beads (Molecular Probes, Eugene, Oreg.) that were labeled with superparamagnetic ferrofluids (Immunicon, Huntingdon Valley, Pa.) as described in U.S. Pat. No. 6,120,856, which is incorporated by reference herein. The magnetic beads were used at a concentration of 3000/mL and were placed in a sample chamber (320 μl) prior to analyzing.

A semi-automated sample preparation system (CellPrep™, as described in U.S. Ser. No. 10/081,996, incorporated by reference herein) was used to process blood samples. In brief, 7.5 mL of whole blood is incubated with EpCAM (epithelial cell adhesion molecule) labeled immunomagnetic particles (ferrofluids described above). The EpCAM antigen is expressed on cells of epithelial origin, but not on blood cells. A series of incubation, separation, and resuspension steps results in a sample of 320 μL placed in a sample chamber (as described in U.S. Ser. No. 10/074,900, incorporated by reference herein) that is held between two poles of a specially designed permanent magnet fixture (as described in U.S. Pat. No. 6,136,182, incorporated by reference herein). The sample contains immunomagnetically-selected cells labeled fluorescently with anti-cytokeratin conjugated to Phycoerythrin (CK-PE), anti-CD45 conjugated to Allophycocyanin (CD45-APC) and the nucleic acid specific dye DAPI (4,6-diamidino-2-phenylindole). The anti-cytokeratin recognizes low molecular weight keratins 4, 6, 8, 10, 13, and 18, present in cells of epithelial origin. The anti-CD45 identifies leukocytes and DAPI stains the cell nucleus.

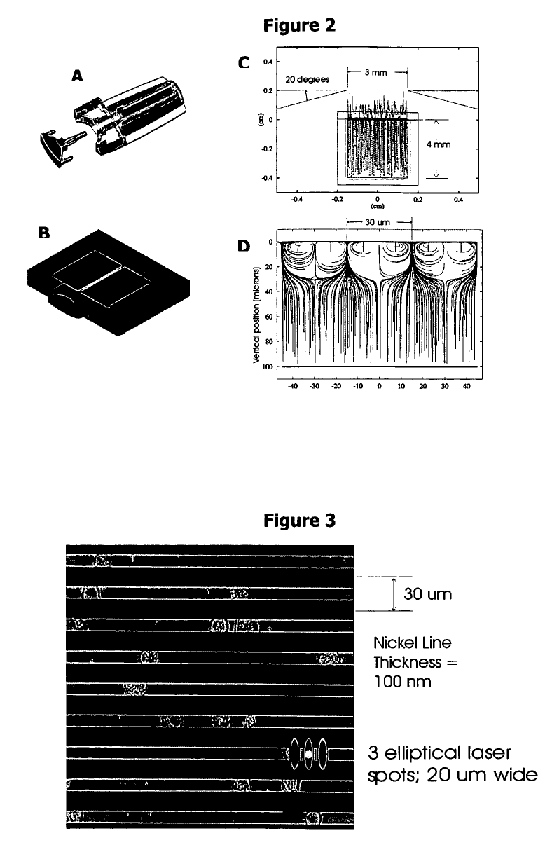

The sample chamber consists of a molded polystyrene housing on which a glass coverslip is attached that bears a series of parallel thin film Nickel lines (Metrigraphics, Wilmington, Mass.). The glass top is affixed to the chamber body with an optical grade UV curable adhesive (Dymax, Torrington, Conn.) thus forming the chamber cavity. The dimensions of the chamber, 30 mm×4 mm×2.7 mm, yield an internal fluid volume of 324 □L. The sample is dispensed into the chamber through an entry port and capped off by the operator to seal out air and allow the chamber to be placed on the instrument for analysis. Computer simulation was used to determine the optimal magnet angle and distance of the sample chamber surface to obtain the most uniform field gradient. When the cells are within reach of the field gradient exhibited by the Nickel lines, they are drawn in between the lines where they are held in place for scanning. FIG. 2A shows the sample chamber and the magnet yoke assembly that holds the chamber between the two magnets is shown in FIG. 2B. For uniform cell distribution, it was necessary to determine the optimal angle of the magnets and the optimal position of the chamber with respect to the two angular shaped magnets, as described in U.S. Pat. No. 6,136,182.

A computer program was written to simulate the movement of magnetically labeled cells in the chamber. The objective was to move all magnetically labeled cells to the upper surface of the chamber but prevent movement to the magnet poles. The distance from the chamber surface to the surface of the magnet must also be short enough to permit viewing with the CD objective that has a working distance of 3.5 mm. FIG. 2C shows such a simulation. The chamber is outlined between the North (N) and South (S) pole of the magnets and the dashed lines indicate the trajectory of magnetically labeled cells. FIG. 2D shows a magnification of the trajectory within the chamber as well as the alignment trajectory when the cells are near the Nickel lines. Additionally, the magnetic fixture provides further enrichment of the sample by separating the magnetically bound material from non-magnetic constituents in the 320 □L sample.

The optical system must provide five functions:
1. hold track alignment in the gap between the Nickel lines,
2. maintain focus on the plane of the cells,
3 & 4. excite and detect fluorescence of the aligned cells, and
5. image selected cells.

FIG. 1 shows the optical system that consists of three lasers for excitation and four photomultipliers for detecting the fluorescent signals from the various fluorescent labels. The three laser beams are combined by dichroic beamsplitters and focused by a CD objective onto the plane of the Nickel lines. Each laser includes anamorphic beam shaping optics to create an elongated focused laser spot at the plane of the cells. The three laser spots are spaced apart with major axis orientation perpendicular to the Nickel lines as shown in FIG. 3.

A magnetic actuation support for the CD objective maintains alignment by moving the objective along two axes, along the optical axis for focus and perpendicular to the Nickel lines for tracking. The 532 nm laser reflection from the Nickel lines is detected through a quarter-wave plate and polarizing beamsplitter by tracking and focus detectors, which provide servo signals for actuation of the CD objective. The system aligns the chamber in preparation for scanning and positions the sample so that the CD objective is aligned on the first pair of Nickel lines. The sample is then moved by a stepper motor in the y-direction (0.1 □m stepsize) along the length of the sample chamber while the CD objective and servo electronics maintain proper focus and tracking. At the end of each line, the instrument indexes the sample in the x-direction by a stepper motor (0.1 □m stepsize) to the next pair of adjacent Nickel lines and repeats the y-direction scanning in the opposite direction. The process is repeated until all 90 Nickel line pairs have been scanned. At 10 mm/sec, the time required to scan all 90 lines is 4.5 minutes.

As each cell passes through a laser spot, the fluorescence is measured by the photomultipliers. Each photomultiplier collects light through a filter and a pinhole, which is parallel to the plane of the Nickel lines. Each pinhole eliminates reflected light from the Nickel lines and views only fluorescence from a selected laser. Minimal crosstalk between fluorescent signals is ensured by the spectral and spatial separation provided by each filter and pinhole. The fluorescence signals are converted to analog signals by a 16-bit analog-to-digital converter board. The multiplexed signals are sampled at a sampling frequency of 25 kHz for a speed in the y-direction of 10 mm/sec. For an epithelial cell with a typical diameter of 12 □m, this sampling rate corresponds to 30 data points across the cell.

To revisit and obtain an image of a specific measured event, it has to be relocated on the sample for which the location in x-y coordinates is needed. To obtain positional information in the y-direction, the stage, which moves the sample under the CD objective, has been equipped with a quadrature encoder (Reneshaw, Gloucestershire, UK) that has a resolution of 0.8 microns. The encoder signals are connected to a counter present on the same analog to digital converter board that samples the photomultiplier signals. Fluorescent signals and y-position information are recorded simultaneously during scanning. The discrete line number on which a specific event is recorded and the positional information in the x-direction is also recorded. In summary, the focus and track system operates as follows:

1. find the origin of the first line;

2. lock focus and track;

3. scan the first line Y-stage 5-10 mm/sec;

4. unlock track and focus;

5. move X-stage to the next line;

6. lock focus and track; and

7. scan the next line.

Steps 4-7 are continued for each line until the entire sample has been scanned. The total scan is 90 lines, times 30 mm/line, which results in a 2.7 m scan for each sample.

Images of selected cells are digitized with a CCD array using the CD objective and imaging optics. To avoid the additional cost, low intensity, and short lifetime associated with broadband light sources, fluorescent imaging is accomplished with the lasers. To avoid laser speckle, an angular scanning mirror scans the laser spots, over the cell as the CCD integrates emitted photons. Using this scanning technique, even very dim fluorescent objects are imaged with excellent signal to noise and resolution.

### EXAMPLE 1

### Determining the Nickel Line Spacing

To assess the size and shape of circulating tumor cells, blood samples of cancer patients were prepared with CellPrep™ and analyzed by a fluorescence microscope system. More than 1000 circulating epithelial cells from 8 patients with a variety of cancers were obtained and cell diameters were measured. Circulating epithelial cells within and between patients were heterogeneous in size and shape. FIG. 5 shows the cell size distribution ranging from approximately 5-30 μm with a mean diameter of 11.3 μm. This posed a problem of selecting a standard line spacing that could accommodate this variation in diameter. If the Nickel lines are spaced to accommodate the larger cells, then there is the risk that smaller cells will occupy the same lateral space or form clusters and be seen as a single event when scanned in the y-direction. If too narrow a space, then the Nickel lines will obscure a large percentage of the cells and compromise imaging of the cell. Criteria were selected for the lines to allow for >95% of the population to be >85% visible.

These criteria were used to establish a line spacing of 15 μm. With a chamber width of 2.7 mm and a 15 μm width of the Nickel, a total of 90 lines are on the chamber surface. The traced fluorescence signals of the objects, and images obtained after revisiting the objects, are analyzed to identify events that are smaller and laterally aligned or clustered between the lines.

A large dynamic range of circulating tumor cells (CTC) ranging from 1 to 5,000 cells per 7.5 mL sample was observed in patient samples. Leukocytes carried over through the sample preparation procedure from 57 blood samples ranged from 428-17,718 cells with a mean of 5,203 and a median of 1,857 leukocytes. The 90 lines on the chamber surface provide a linear space of 2.7 meters for capturing cells. Assuming an average tumor cell size of 12 μm and allowing an occupancy of 10%, the chamber capacity is 22,500 tumor cells and is well within the dynamic range of both captured tumor cells and leukocytes.

### EXAMPLE 2

### Tumor Cell Analysis by the System

Cells from the prostate carcinoma tissue culture cell line, PC3, were used at a concentration of 5,000 PC3 cells/mL. Aliquots (10-100 □L) of this cell suspension were spiked into 7.5 mL whole blood samples of normal donors to obtain blood samples with low tumor cell numbers.

Approximately 300 PC3 cells were spiked into 7.5 mL of blood and processed with CellPrep™ as described above. When the sample is dispensed into the sample chamber while being held in the magnetic field, the immunomagnetically labeled cells are drawn to the upper inside surface of the chamber by magnetic forces from the permanent magnets. A bright field image of the aligned PC3 cells is shown in FIG. 3.

FIG. 6 shows the analysis of the sample. The 2-dimensional dot plot in FIG. 6A shows the fluorescence signals of CK-PE and CD45-APC. The tumor cell candidates (PC3-cells) staining with CK-PE and lacking CD45-APC can be clearly discriminated from the leukocytes staining with CD45-APC and lacking CK-PE. Events that stain with cytokeratin as well as CD45 are tumor cells that stain non-specifically with CD45, leukocytes that non-specifically stain with Cytokeratin or debris. To verify that events in the region typical for tumor cells are indeed cells the waveforms of the fluorescence signals are analyzed. FIG. 6B shows the waveform of the CK-PE signal for one of the events within the tumor cell region. The size of the event is 20 μm as provided by the encoder position along the line in concert with the fluorescence signal. The distribution of the cytokeratin throughout the cytoplasm shows a demarcation in the first part of the signal that coincides with the nucleus of the cell. If the waveform of this particular event strongly suggests that this indeed is a cell, the system relocates to this position in the chamber to obtain bright field and nuclear images of the object. The sample is illuminated with a blue LED positioned below the sample chamber and the CCD captures a bright field image of the object. The bright field image shown in FIG. 6C clearly shows the typical features exhibited by a cell. To verify that the cell indeed contains a nucleus, an image of the nuclear staining of DAPI is obtained. The scanning mirror (FIG. 1) distributes the light of the violet laser with an angle scanning range of 5 mrad at a frequency of 500 Hz over the location of the object and an image is taken with the CCD camera. The fluorescent image of the DAPI is shown in FIG. 6D. The nucleus clearly is contained within the outline of the cell shown in FIG. 6C.

### EXAMPLE 3

### Scanning Methods

In the present instrument system, cells are imaged with laser illumination to avoid the additional cost, low intensity, and short lifetime of broadband light sources. However, the long coherence length of lasers and light reflections in the imaging system contribute coherent noise and speckle in the image. Also, the Gaussian intensity profile of the laser beam does not provide uniform illumination of the object. Both of these problems with coherent illumination are overcome by moving the laser spot across the object as the CCD array integrates the light. Movement of the source during exposure washes out the coherent noise in the image. Any angular scanning means, such as a scanning mirror, rotating mirror, electro-optic or acousto-optic deflector, or electro-refractive device could be used to deflect the beam by a small angle, providing motion of the focused laser spot at the object plane. By driving this device with a periodic signal, the spot is scanned back and forth over the object.

One example of this concept is shown in FIG. 1, which shows the optical configuration for the instrument. Three lasers illuminate the magnetically confined object and four PMT detectors measure fluorescence from the object as it passes through each laser spot in the focal plane of the objective lens. The three laser beams are combined by dichroic beamsplitters and focused by a CD objective onto the plane of the nickel lines. Each laser includes anamorphic beam shaping optics to create an elongated focused laser spot at the plane of the cells. The three laser spots are spaced apart with major axis orientation perpendicular to the nickel lines. A magnetic actuation support for the CD objective maintains alignment by moving the objective along two axes, along the optical axis for focus and perpendicular to the nickel lines for tracking. The 532 nm laser reflection from the nickel lines is detected through a quarter-wave plate and polarizing beamsplitter by tracking and focus detectors, which provide servo signals for actuation of the CD objective.

As each cell passes through a laser spot (illumination means), a corresponding PMT (detection means) measures the fluorescence. Each PMT collects light through a filter and a pinhole, which is conjugate to the plane of the nickel lines. Each pinhole eliminates reflected light from the nickel lines and views only fluorescence from a selected laser. Minimal cross talk between fluorescent signals is insured by the spectral and spatial separation provided by each filter and pinhole.

During the imaging step, the object is moved into a laser spot, which is scanned back and forth by a scanning mirror. The total angular scan range is appropriate for the size of illumination region and focal length of the objective. The scanning mirror reflects the laser beams through a beamsplitter and objective lens to the object plane. The fluorescence or scattered light from the object passes back through the same objective lens and beamsplitter to a second lens and CCD array for imaging. The scanning frequency must be sufficient to provide many full scans during the integration time of the CCD array. If the mirror passes through only a few full scans and one partial scan, the integrated illumination over the object will not be uniform. The CCD should only collect photons during an integral number of full scans, or many full scans plus one partial scan. Then the partial scan contribution becomes a small portion of the total integrated current on each CCD pixel.

After the imaging scans are completed, the optical system will scan more tracks to measure other fluorescent objects with the PMT detectors. Therefore, the static position of the scanning device must return the system to the original optical alignment of the PMT pinholes with the laser beams and nickel line gap. The system shown in FIG. 1 ensures this alignment by placing the scanning device and CCD array after the 3 band filter which splits the lasers and fluorescent beams going to the PMT detectors. Variations of the scanning mirror static position between successive imaging sessions will not effect the alignment of pinholes with the laser spots and nickel lines.

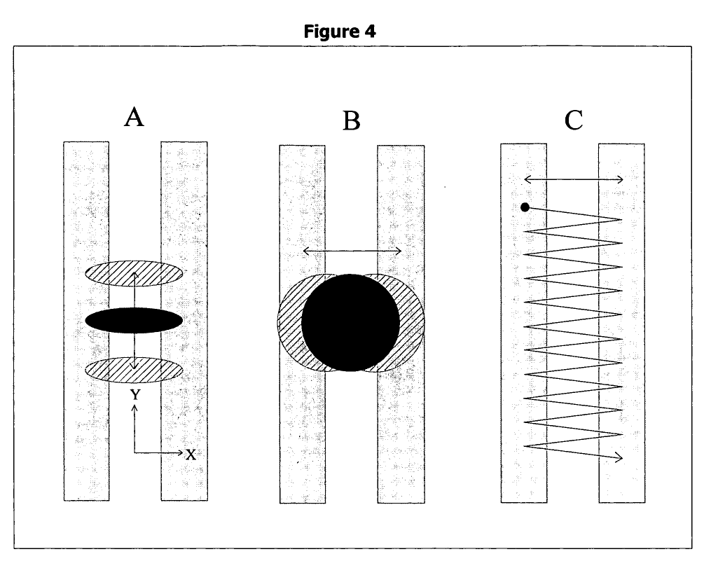

FIG. 4 shows two configurations for laser scanning. The instrument system uses an elongated laser spot, which spans adjacent nickel lines, to excite fluorescence of objects between the lines and to provide tracking and focus servo signals from light reflected by the lines. In FIG. 4A, this elongated laser spot is scanned along the Y-axis to illuminate an extended region between the nickel lines. Illumination uniformity is maintained by scanning over a distance that is larger than width of the laser spot. Due to the high intensity of the laser spot, even very dim fluorescent objects are imaged with excellent signal to noise and resolution by using this scanning technique.

In FIG. 4B the object plane is moved out of the focal plane of the laser to increase the illumination area and intensity uniformity over the region of interest. The scanning mirror moves this broad spot back and forth across the object, eliminating coherent noise and structure in the image. The broad beam, in FIG. 4B, covers a large region when scanned in either the X or Y direction. In both FIGS. 4A and 4B, the coherent noise in the image may be reduced to acceptable levels with very small motion of the spot. However, removal of the spot intensity profile requires a larger scan amplitude. As before, the CCD must integrate over many scans of the mirror to reduce the effect of a partial start or stop scan. The total of all scans must approach an integral number of scans as the scan number decreases to maintain image uniformity.

In addition to imaging, the scanning technique could improve the resolution of the fluorescent signals from the PMT detectors. As the laser beam in FIG. 4A moves across a cell, it illuminates a large portion of the cell at any time. However, the fluorescent signal cannot accurately represent the small structural details of the cell. The Fourier spectrum of the nuclear stain fluorescent signals may differentiate between normal and tumor cells. To obtain the high frequencies of the cell structure requires interrogation of the cell with a small spot to provide resolution of the smaller components of the cell. A single scan of a small spot through the middle of the cell would provide high frequency response only over a small portion of the cell. By scanning a small laser spot in the X direction, while the cells are moving in the Y direction, a larger portion of the cell is interrogated by a raster pattern of the exciting beam as shown in FIG. 4C. Spectral analysis of the signal from the PMT, which views this spot during the raster scan, may provide additional information about the malignant state of the cell. Amplitude variations of this signal also indicate the stage of the cell, or the presence of nucleoli or chromatin structures. A fluorescent image of the cell is then constructed from this signal in the same manner that a television constructs an image from a video signal.

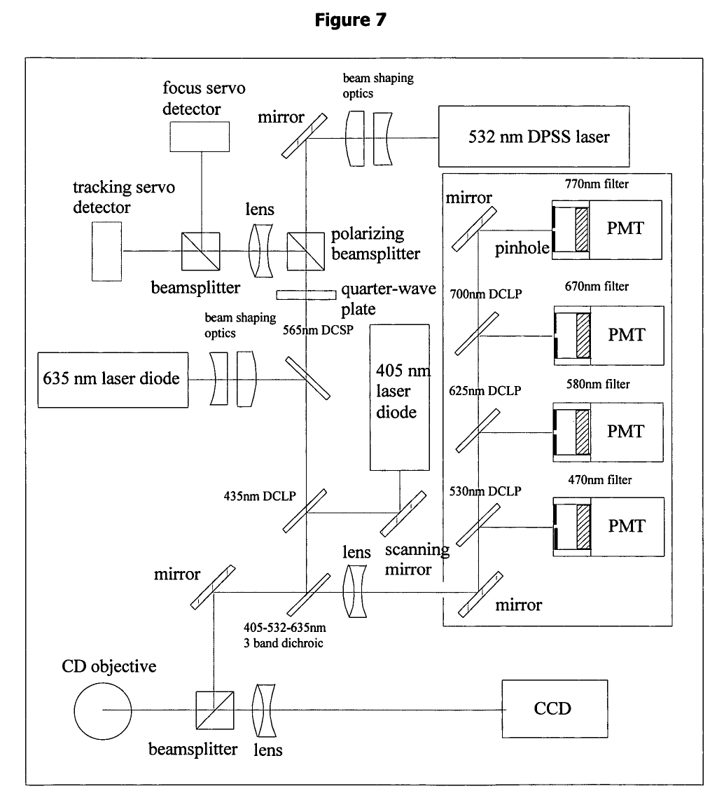

The scanning mirror in FIG. 1 is oriented to scan the laser spot in the X direction and provide the raster pattern. The optical system must remain locked on focus and track during the raster scan, and the spot of the tracking laser should not move in the X direction relative to the nickel lines. The focus and track laser beam must not pass through the scanning or deflection device to conserve servo alignment during the raster motion. The configuration shown in FIG. 7 will allow one or more lasers to scan in the X or Y direction while another static laser provides the signals for the focus and track servos. In FIG. 7, only the 405 nm laser is scanned. A scanning mirror can be placed in the path of any laser, except for the tracking laser, to produce fluorescent images for that laser. The PMT which views the scanning laser spot will produce a raster fluorescent signal for the corresponding excited dye. This configuration also accommodates the CCD image scanning described in FIGS. 4A and 4B for the lasers which pass through the scanning device.

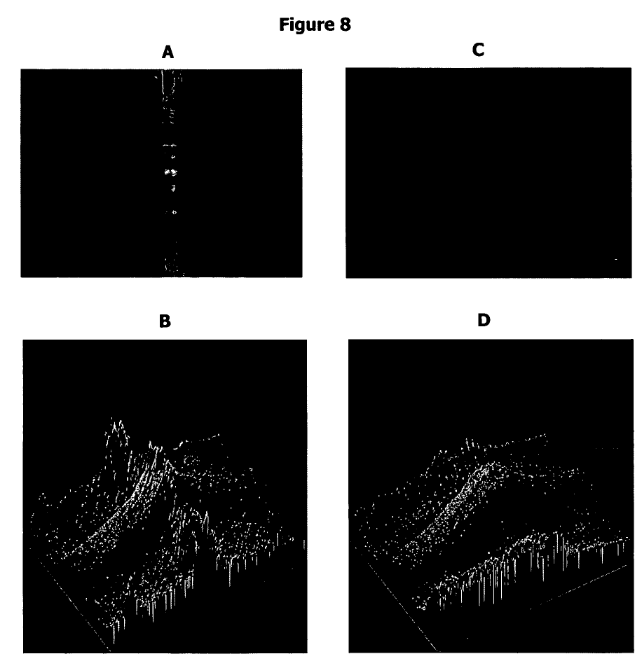

FIG. 8 shows a sample chamber that was loaded with a solution of DNA that was labeled with DAPI. The chamber is illuminated with the 405 nm laser diode. The intensity profiles are speckled and narrow with the static laser, but are corrected with the scanning mirror. This is seen by the differences between 8A, the static laser image and 8B, the corresponding intensity profile, and 8C, the scanning laser image and 8D, its corresponding intensity profile. The scanning laser provides more uniform illumination and reduces the speckling effect by averaging the speckle while scanning.

### EXAMPLE 4

### Revisiting Objects of Interest

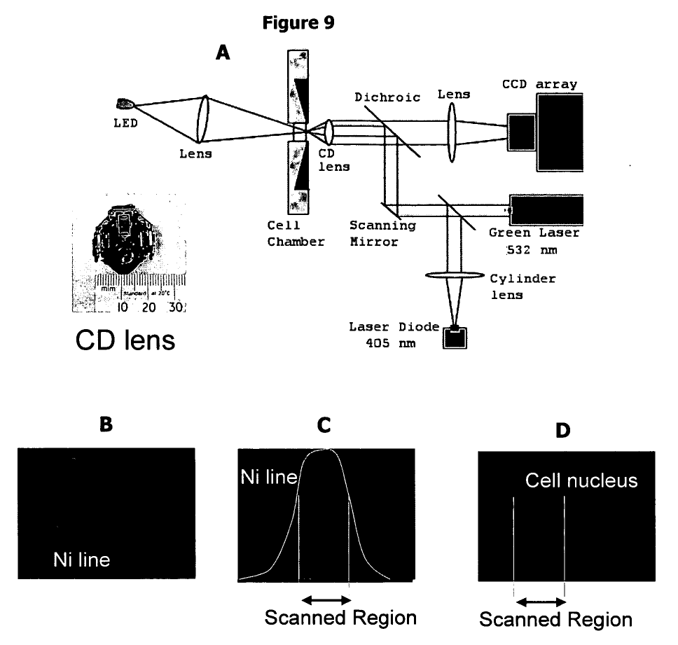

As the sample is scanned and analyzed, there will be objects that are detected which the instrument operator may wish to revisit. Because the system is capable of obtaining data from the sample object as well as its location, the system can easily return to the location of the object for further analysis of the object. This further analysis allows for confirmation of the object's classification by the system. The imaging subsystem that is used for revisiting objects of interest is shown in FIG. 9. The revisiting process uses a bright-field illumination system to further image objects of interest. Briefly, the procedure is:

1. the system returns to the position of interest;

2. the LED which is placed off-axis to enhance contrast is turned on;

3. a bright field image is recorded on the CCD array;

4. the mirror starts raster scanning to provide an uniform laser illumination of the region;

5. a fluorescent image is recorded on the CCD array; and

6. the images are pseudo colored and combined.

These steps can be seen in FIGS. 9B, 9C, and 9D. In 9B, the bright field image is shown, where there are three objects of interest. In these figures, the middle one is the subject of the revisitation analysis. FIG. 9C shows the scanning laser image, which images the nucleus. In FIG. 9D, the bright field and scanning laser images are combined.

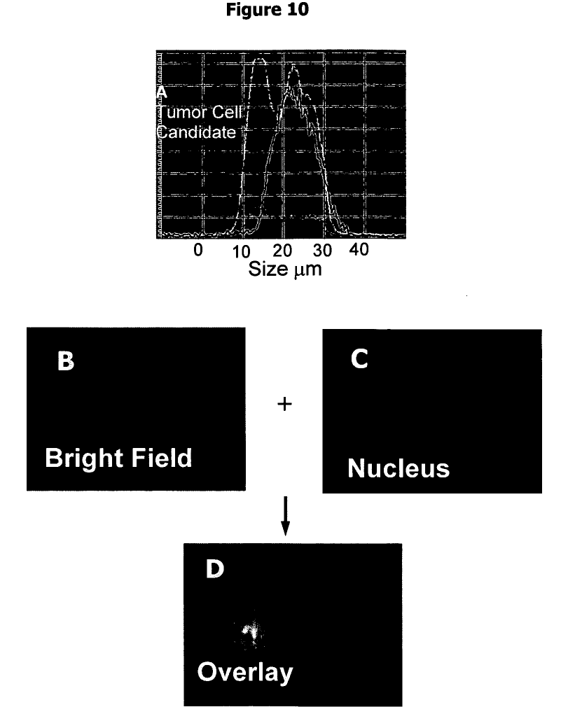

FIG. 10 shows an analysis of the system, which has found a tumor cell candidate (10A). This object is then revisited for further analysis and confirmation. FIG. 10B shows the bright field imaging of the object, using subsystem described above. The scanning laser then images the object for nucleic acid, as shown in 10C. Finally, these two images are combined to give a complete picture of the object.

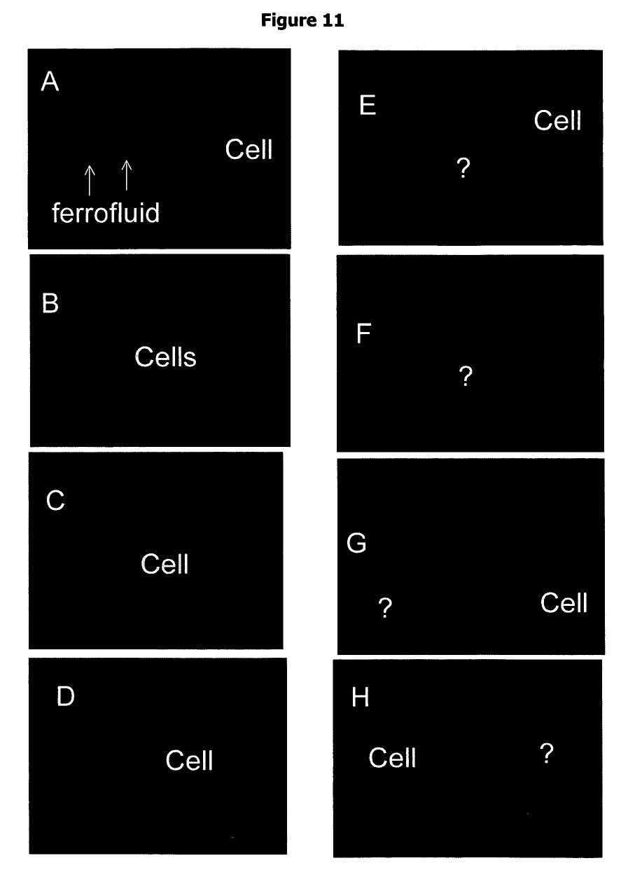

FIG. 11 shows various objects of interest as imaged by the bright field imaging subsystem. Different objects are labeled in each of the panels, pointing out ferrofluid lines, cells, and questionable areas that may or may not be real objects. However, just by viewing these images alone, one would not be able to easily classify them, as described in PCT/US02/26861, which is incorporated by reference herein. The next step would be to use the scanning laser at each of these objects to obtain further information that would be used for classification. By using the imaging system and methods described in this invention, these objects would be classified, providing the instrument operator with valuable information. Multicolor fluorescence intensity analysis permits the identification of rare tumor cell candidates. The addition of fluorescence signatures, bright field image and DAPI image permits the further classification into:

1. single intact or damaged cells,

2. debris or cell fragments,

3. cell clusters or epithelial cells attached to leukocytes, and

4. leukocytes nonspecifically binding to the epithelial cell marker.

The preferred embodiments of the invention as herein disclosed, are also believed to enable the invention to be employed in fields and applications additional to cancer diagnosis. It will be apparent to those skilled in the art that the improved diagnostic modes of the invention are not to be limited by the foregoing descriptions of preferred embodiments. Finally, while certain embodiments presented above provide detailed descriptions, the following claims are not limited in scope by the detailed descriptions. Indeed, various modifications may be made thereto without departing from the spirit of the following claims.

We claim:

1. An apparatus for detecting and analyzing biological entities present in a biological sample, said apparatus comprising:
a. a sample chamber containing said biological sample,
b. one or more illuminating means for illuminating said biological entities present in biological sample,
c. one or more detection means for detecting and imaging said biological entities, and
d. a stage that provides the means to hold and move said sample chamber through areas of illumination.

2. The apparatus of claim 1, wherein said illuminating means is selected from the group consisting of laser illumination, LED illumination, and filtered white light.

3. The apparatus of claim 1, wherein said detection means is selected from the group consisting of a two-dimensional detector array, one or more photomultiplier tubes, and one ore more CCD cameras.

4. The apparatus of claim 1, wherein at least one of said illuminating means is capable of a scanning motion as it illuminates said biological sample.

5. The apparatus of claim 4, wherein said scanning motion produces a raster image.

6. The apparatus of claim 1, wherein said stage is capable of tracking the position of said sample chamber in two dimensions.

7. An apparatus for detecting and analyzing cells present in a biological sample, said apparatus comprising:
a. a sample chamber containing said biological sample,
b. one or more illuminating means for illuminating said cells present in biological sample,
c. one or more detection means for detecting and imaging said cells, and
d. a stage that provides the means to hold and move said sample chamber through areas of illumination.

8. The apparatus of claim 7, wherein said illuminating means is selected from the group consisting of laser illumination, LED illumination, and filtered white light.

9. The apparatus of claim 7, wherein said detection means is selected from the group consisting of a two-dimensional detector array, one or more photomultiplier tubes, and one ore more CCD cameras.

10. The apparatus of claim 7, wherein at least one of said illuminating means is capable of a scanning motion as it illuminates said biological sample.

11. The apparatus of claim 10, wherein said scanning motion produces a raster image.

12. The apparatus of claim 7, wherein said stage is capable of tracking the position of said sample chamber in two dimensions.

13. A method for detecting and analyzing biological entities present in a biological sample, said method comprising:
a. obtaining said biological sample suspected to contain said biological entities,
b. introducing said sample into a sample chamber,
c. illuminating said sample with one or more illumination means,
d. moving said sample chamber through an illuminated area created by said illumination means,.
e. detecting said illumination with one or more detection means to produce a detection signal, and
f. analyzing said detection signal.

14. The method of claim 13, wherein the position of said sample chamber movement is tracked in two dimensions.

15. The method of claim 14, wherein the positional data is used to return to various detection signals for further analysis.

16. The method of claim 13, wherein said illuminating means is selected from the group consisting of laser illumination, LED illumination, and filtered white light.

17. The method of claim 13, wherein said detection means is selected from the group consisting of a two-dimensional detector array, one or more photomultiplier tubes, and one ore more CCD cameras.

18. The method of claim 13, wherein said detection signal is the amount of fluorescence emitted by said cells.

19. The method of claim 13, whereby as said sample chamber is moved through said illuminated area, said one or more illumination means is moved in a scanning motion to form a rastered illumination.

20. The method of claim 19, wherein the analysis of said detection signal includes reconstructing said detection signal from said rastered illumination to provide detailed information of said biological entities.

21. The method of claim 13, wherein said biological sample is selected from the group consisting of: peripheral blood, bone marrow, leukopheresis, tissue homogenates, nipple aspirates, colonic lavage, sputum, bronchial lavage, alveolar lavage, pleural fluids, peritoneal fluids, pericardial fluids, and urine.

22. The method of claim 13, wherein said biological entities are selected from the group consisting of: hormones, proteins, peptides, lectins, oligonucleotides, drugs, chemical substances, nucleic acid molecules, cells, viruses, and bacteria.

23. A method for detecting and analyzing cells present in a biological sample, said method comprising:
a. obtaining said biological sample suspected to contain said cells,
b. introducing said sample into a sample chamber,
c. illuminating said sample with one or more illumination means,
d. moving said sample chamber through an illuminated area created by said illumination means,
e. detecting said illumination with one or more detection means to produce a detection signal, and
f. analyzing said detection signal.

24. The method of claim 23, wherein the position of said sample chamber movement is tracked in two dimensions.

25. The method of claim 24, wherein the positional data is used to return to various detection signals for further analysis.

26. The method of claim 23, wherein said illuminating means is selected from the group consisting of: laser illumination, LED illumination, and filtered white light.

27. The method of claim 23, wherein said detection means is selected from the group consisting of a two-dimensional detector array, one or more photomultiplier tubes, and one ore more CCD cameras.

28. The method of claim 23, wherein said detection signal is the amount of fluorescence emitted by said cells.

29. The method of claim 23, whereby as said sample chamber is moved through said illuminated area, said one or more illumination means is moved in a scanning motion to form a rastered illumination.

30. The method of claim 29, wherein the analysis of said detection signal includes reconstructing said detection signal from said rastered illumination to provide detailed information of said cells.

31. The method of claim 23, wherein said biological sample is selected from the group consisting of peripheral blood, bone marrow, leukopheresis, tissue homogenates, nipple aspirates, colonic lavage, sputum, bronchial lavage, alveolar lavage, pleural fluids, peritoneal fluids, pericardial fluids, and urine.

32. The method of claim 23, wherein said cells are selected from the group consisting of rare cells, fetal cells, stem cells, circulating tumor cells, circulating epithelial cells, and circulating endothelial cells.
